(19) United States
(12) Patent Application Publication
(10) Pub. No.: US 2009/0110654 A1
(43) Pub. Date: Apr. 30, 2009

(54) Bio-Plastic Composite Material, Method of Making Same, And Method of Using Same

(76) Inventors: Ronald T. Hagemann, Edgerton, WI (US); Daniel D'Amico, Tuckahoe, NY (US)

Correspondence Address: Buchanan Ingersoll & Rooney P.C.;One Oxford Centre, 20th Floor, 301 Grant Street, Pittsburgh, PA, 15219-1410, US

(21) Appl. No.: 12/127,855

(22) Filed: May 28, 2008

Related U.S. Application Data
(60) Provisional application No. 60/983,387, filed on Oct. 29, 2007.

(51) Int. Cl. A61L 9/013; A61L 9/01; B29B 9/12

(52) U.S. Cl. 424/76.1; 424/76.5; 523/102; 264/6

(57) ABSTRACT

A bio-plastic composite comprises at least one biological material and one plastic material. The biological material in the bio-plastic composite is hydrolyzed, classified, or cryogenically ground for enhanced integration with the plastic material. A bio-odor generated during process of making bio-plastic composites is counter acted or masked by integrating odor controlling agents into bio-plastic composites.

### CROSS-REFERENCE TO RELATED APPLICATIONS

This application claims priority to pending U.S. Provisional Patent Application No. 60/983,387, filed on Oct. 29, 2007, and having a common inventor with this application. The entire contents of that application are incorporated by reference herein.

### FIELD OF THE INVENTION

This invention generally relates to composite materials. More specifically to bio-plastic composite materials in which biological material is integrated with plastic material. The invention also relates to odor controlled bio-plastic composites and manufacturing of bio-plastic composite pellets.

### BACKGROUND OF THE INVENTION

The use of composite materials in all products, from sporting goods, to aviation products, to structural support materials is increasing. Composite materials comprise two or more materials combined in such a way that the individual materials are distinguishable. Monolithic material on the other hand means the material typically consists of a single material such as glass or plastic, or in some cases a combination of materials that are indistinguishable such as a metal alloy.

Composite materials which include carbon and/or glass fiber reinforced structures are now readily available in the market. Composite materials offer the opportunity for comparable or better strength and stiffness characteristics typically at a mere fraction of the weight. Composite materials also offer opportunities for providing far superior corrosion resistance and insulating and thermal barrier properties than steel, metal, or wood.

The individual materials that make up a composite material are typically called constituents. Traditionally, composites basically comprise at least two constituent materials including a binder (what is commonly referred to in the industry as forming the “matrix”); and a reinforcement. The reinforcement is usually stronger and provides for stiffness as compared with the matrix. The reinforcement defines in large part the composite material properties. The matrix holds the reinforcement in an orderly pattern, which may be flat, curved or profiled. The matrix helps to transfer loads among the different fibers and plies of the reinforcement materials. Typically and by design the matrix which transfer loads very short distances while the reinforcement bears loads over longer distances.

Reinforcement materials usually comprise one or more types of fiber material to include discontinuous fiber and continuous fiber. The most common materials for the reinforcements as applied to typical composite materials include: fiber glass and carbon fiber. Additionally, various bio-fibers are proposed in U.S. Patent Publication No. US 2005/0013982 to Burgueno et al. Fibers may be woven into a cloth or mat and thus bi-directional (providing support among more axes) or arranged in a “unidirectional” manner in a single ply either randomly or in a predetermined arrangement. Reinforcements may also include plastic materials, metal materials and glass fiber reinforced plastic.

Matrix materials are usually some type of petroleum based plastic resins. Resins are liquid polymers that can fill in the spaces around the reinforcements that when catalyzed will cure to a solid. Common plastic resin type matrices include for example polyurethane, polypropylene, polyethylene, polyvinyl chloride, epoxy, polyester, polyether, vinyl ester and other suitable types of resins. While synthetic petroleum based resins are typical, there is also known bio-based resins such as isocyanate (e.g. PMDI) and polyol soybean oil such is believed to be known in the art.

While reinforcements and matrix materials are the primary constituents of a composite material, there are also other materials which may be added which are used to modify the properties of the polymeric resins which make up the matrix. Categories of additives include reagents, fillers, viscosity modifiers, pigments and others. Fillers for example are materials which may be added to the resin to vary the properties and/or extend the volume of the matrix. Other additives such as accelerators are used to control the rate at which curing can occur. Gel coats are also used typically on the outside surface of a composite. The gel coat may include a different polyester resin that may be colored or clear to provide a cosmetic and weatherability enhancement.

While composite materials have found wide use in many higher end industries such as aircraft, wind-turbine, sporting goods and medical, the applications of composites across industry have been somewhat limited. This may be due in part to cost issues relating to existing methods of composite production as well as the cost of the input materials. Composite materials often rely heavily on petroleum based resin products, which not only is disadvantageous from a cost standpoint, but also an environmental standpoint. Petroleum reserves are also not an unlimited resource and attempts to reduce oil imports and/or oil use is desirable. Attempting to provide excellent strength and low weight properties in a composite to those of typical monolithic materials can be a challenging task while making the material in a practical and economic cost effective manner. Not surprisingly, there have been several attempts at providing solutions to these issues and some progress has been made.

One area of research in composite materials has been related to incorporating natural fibers and agricultural material into composites. The U.S. patent application Ser. No. 11/492,470, filed by the same inventor of the present invention teaches composite material with grain filler and method of making the same, the entire disclosure of which is hereby incorporated by reference. Others have taught methods of processing cellulous agricultural materials. U.S. patent application Ser. No. 10/494,646, published on Aug. 11, 2005, teaches a method of processing ligno-cellulosic material using hydrothermal pressure vessel, the entire disclosure of which is incorporated by reference. This method includes steps of comminuting of the material, drying, subjecting the material packed vessel to stream under pressure, and then drying the processed material to a specific moisture content. This method is referred to as LignoTech and may be utilized as one method of preparing a biological material to be integrated with a plastic material and an odor controlling agent in some embodiments of the present invention. There continues to be a desire for further improvements in the composites industry for which the present invention is directed.

### BRIEF SUMMARY OF THE INVENTION

The present invention is directed toward bio-plastic composite material and method of making the same where at least one plastic material is integrated with at least one processed or unmodified biological material. The methods of processing biological material according to the present invention may involve hydrolysis, classification, and/or cryogenic grinding. Certain benefits from processing biological material according to the present invention include ability to increase amount of the biological material in a bio-plastic composite, and enhanced composite properties. With a hydrolysis process, up to 99%, by weight, biological material may be integrated into a bio-plastic composite. The classification process allows for selection of biological material with a specified particle size range, thereby enhancing strength and stiffness characteristics and improving consistency of bio-plastic composite properties.

In one aspect, the present invention provides odor controlled bio-plastic composite material and method of making the same. Biological materials generate a bio-odor during process of making bio-plastic composite material. This can occur due to natural decay, oxidation, and/or processing and or other odors derived from the process and or raw materials. This odor may linger significantly following manufacture of the material. The bio-odor is usually considered as a malodor rendering some bio-plastic composites unmarketable. In this aspect of the present invention, an odor controlling agent, alone or in combination with anti-oxidants and hindered amines is integrated in bio-plastic composites to counter act or mask the bio-odor. Methods of integrating the odor controlling agent at various stages of the process of making bio-plastic composite material are discussed.

In another aspect, the present invention provides for various manufacturing processes of making bio-plastic composites. These manufacturing processes may involve extrusion, injection molding, injection blow molding, compression molding, coextrusion, and/or thermoforming. For example, one embodiment of the invention provides a method of making bio-plastic composite comprising steps of reducing a biological material to a particle size appropriate for a hydrolysis process; drying the sized biological materials to a moisture content less than 25% by weight; hydrolyzing the dried biological material in a pressurized hydrothermal vessel by subjecting the biological material with steam; drying the hydrolyzed biological material to a moisture content less than 15% by weight; and forming a bio-composite by integrating the dried hydrolyzed biological material with a plastic material.

Another embodiment of the invention provides a method of making bio-plastic composite comprising steps of processing a biological material; classifying the biological material to separate a fiber material from a non-fiber material; selecting the classified fiber material according to a desired composite property; and forming a bio-plastic composite by integrating the selected fiber material with a plastic material. In a further embodiment the method of making the bio-plastic composite includes wherein the the biological material is at least one of DDT and other byproducts of energy production generated from at least one of the group consisting of corn, soybean, flaxseed, switchgrass, rapeseed, miscanthus, hulls, stover, straw, bagasse from sugarcane and jatropha processed using a hydrolysis system. The biological material may be ground.

Another embodiment of the invention provides a method of making bio-plastic composite comprising the steps of freezing a material including a biological material using a cryogen; shattering the frozen material to form a powdered material; and integrating the classified powdered material with a plastic material to form a bio-plastic composite. In a further embodiment the frozen material includes at least one of a recycled tire material and a recycled high temperature plastic material, and at least one biological material. In a further embodiment the method includes wherein the cryogen is a liquid nitrogen at about negative 320° F. The powdered material may be classified to a desired particle size range using a screen with an appropriate mesh size, then integrated with a plastic material to form a bio-plastic composite.

Other aspects, objectives and advantages of the invention will become more apparent from the following detailed description when taken in conjunction with the accompanying drawings.

### BRIEF DESCRIPTION OF THE DRAWINGS

The accompanying drawings incorporated in and forming a part of the specification illustrate several aspects of the present invention and, together with the description, serve to explain the principles of the invention. In the drawings:

FIG. 1 is a perspective view of a bio-plastic composite material member with cross section being taken through one end, in one embodiment of the present invention where classified biological material is integrated with a plastic material, with biological material particles magnified out of proportion to show consistency in particle size distribution;

FIG. 2 is a perspective view of a bio-plastic composite material member with cross section being taken through one end, in one embodiment of the present invention where hydrolyzed or cryogenically ground biological material is integrated with a plastic material, with biological material particles magnified out of proportion to show consistency in particle size distribution;

FIG. 3 is a schematic representation of a method of processing a biological material using a air sieving classification system according to one embodiment of the present invention;

FIG. 4 is a partially schematic illustration illustrating a method of making bio-plastic composite pellets according to one embodiment of the present invention;

FIG. 5 is a partially schematic illustration illustrating a method of making an odor controlled bio-plastic composite according to one embodiment of the present invention.

FIG. 6 is a partially schematic illustration illustrating a method of manufacturing a bio-plastic composite using an injection molding system.

FIG. 7 is a perspective view of bio-plastic composite samples according to one embodiment of the present invention.

FIG. 8 is a process flow diagram of method of making an odor controlled bio-plastic composite according to one embodiment of the present invention.

FIG. 9 is a process flow diagram of a method of making an odor controlled bio-plastic composite according to another embodiment of the present invention.

FIG. 10 is a process flow diagram of a method of making an odor controlled bio-plastic composite according to yet another embodiment of the present invention.

FIG. 11 is a process flow diagram of a making an odor controlled bio-plastic composite according to one embodiment of the present invention.

FIG. 12 is a process flow diagram of a making an odor controlled bio-plastic composite according to another embodiment of the present invention.

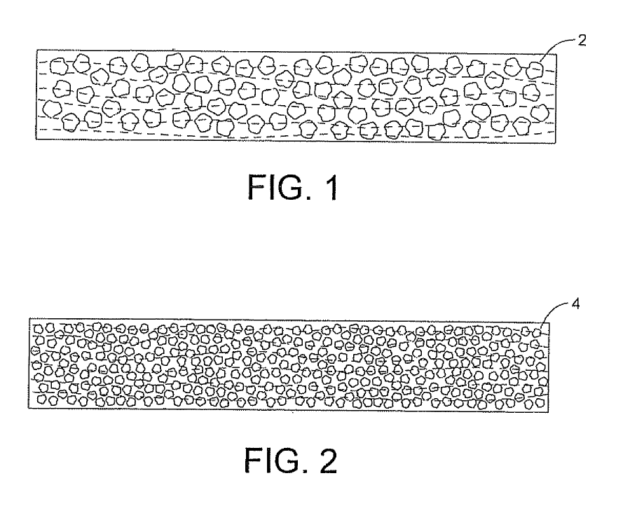

While the invention will be described in connection with certain preferred embodiments, there is no intent to limit it to those embodiments. On the contrary, the intent is to cover all alternatives, modifications and equivalents as included within the spirit and scope of the invention as defined by the appended claims.

### DETAILED DESCRIPTION OF THE INVENTION

The following disclosure will detail particular embodiments according to the present invention, which provides bio-plastic composite materials, and methods of making the same. First, constituents of bio-plastic composite materials are described. Then methods of preparing the constituents, particularly, methods of processing biological material are explained. Finally, various methods of manufacturing bio-plastic composites are provided.

Bio-Plastic Composite Constituents

The bio-plastic composite materials of the present invention include at least one biological material, at least one plastic material, and may include one or more of odor controlling agents or additives.

Biological Materials

The biological material in the bio-plastic composite materials may constitute any suitable agricultural grain including, for example: corn, soybean, wheat, barley, oats, sorghum (milo), sunflower, safflower, buckwheat, flax, peanut, rice, rape/canola, rye, millet, triticale, chickpeas, lentils, and field peas and/or harvestable flower portion of a plant. All parts of grain crop plants (any shell, leaf, stalk, or trunk) such as corn stalks, corn cobs, and rice hulls are excellent candidates for the purpose of this invention. The biological material also may constitute agricultural wastes including but not limited to cereal straw, sawdust, woodchips, waste wood particulates, bark, newsprint, other paper and card board. Moreover, biological fibers such as fibers from kenaf, switchgrass, hay, straw, bagasse from sugarcane, jatropha, and other similar plants are included in the scope of this invention. The biological materials may be a refined product (e.g. starch or flour), a waste byproduct from grain processing, and or simple ground up grain products.

One preferred biological material for embodiments is the byproducts of ethanol or other alcohol production which include both byproducts of wet milling and dry milling. Distillers dried grain (DDG) is a common byproduct of dry milling ethanol production. Over 34 billion pounds of DDGs are created in domestic dry milling ethanol production today. For every bushel of corn made into ethanol, 18 pounds of DDGs are created. The corn kernel is mostly starch at 61% of the wet weight, with protein, fiber, corn oil, and water making up the remaining 39%. The dry milling ethanol process uses most of the starch present in the corn kernel during ethanol fermentation.

Dry grind ethanol production begins by grinding corn into a coarse flour and combining with water and enzymes. The enzymes begin the conversion process of starch to sugar crating a mash that is then cooked and sterilized. After cooling, yeast is mixed with the mash to ferment the sugars into ethanol, carbon dioxide and other metabolites. The fermented mash is then sent to distillation to extract the ethanol. The mash is now considered spent mash which then goes into either a screen press or centrifuge, where most liquid in the mash is separated.

The spent grains can be sold as wet distillers grains or dried to be sold as distillers dried grains (DDG). These distillers grains may be sold as livestock feed. However, the tremendous growth in fuel ethanol production has greatly increased the supply of distillers grains, flooding the market. Therefore, distillers grains are in large supply and have relatively low value, thus they are often considered to be a waste product from ethanol production. Thus, incorporating distillers grains in the bio-plastic materials provides for both environmentally friendly and economically beneficial alternative to traditional composite materials.

The biological material processing methods of the present invention also makes it possible to utilize the byproducts of wet milling production (wet mills). Today, ethanol plants are faced with higher cost to dry the wet mills than what they can sell the dried mills in the market. Therefore, use of the wet mills in the present invention, can prevent the wet mills from becoming waste products and reduce cost of the bio-plastic composite material by introducing a low cost constituent. Considering that about one in six rows of corn in the United States are dedicated to ethanol production, the present invention provides an advantageous and beneficial use for such byproduct of ethanol production.

In addition to energy production byproducts formed from agricultural grain, it has been recognized that ethanol or other bio-based energy production can incorporate other parts of the plant, to include the foliage (leaves, stems etc). As a result, the present invention also is intended to cover other byproducts of bio-based energy production from biological material. The biological material constituent may also be formed from any such biological material byproducts of energy production to include the foliage from corn, soybean, flaxseed, switchgrass, rapeseed, miscanthus, stover, hay, straw, bagasse from sugarcane, jatropha, or other such foliage crop which is used in bio-based energy production. Such foliage can be processed with the grain in energy production. Thus, as used herein, byproducts of bio-based energy production from biological material and other similar terms is meant to include energy production from grains and/or foliage. Corn oil extracted from ethanol production may be used as a biological material constituent.

Raw Plastic Materials

Suitable plastic materials for use in embodiments include both addition polymer and condensation polymer materials such as melamine polyolefin, polyacetal, polyamide, polyester, cellulose ether and ester, polyalkylene sulfide, polyarylene oxide, polysulfone, modified polysulfone polymers and mixtures thereof. Preferred plastic materials that fall within these generic classes include polyethylene, polypropylene, polyurethane, polyvinyl chloride, epoxy, polyester, polyether, vinyl ester and polyamide. While synthetic petroleum based resins can be used and are within the scope of the present invention, a preferred resin for environmental and petroleum conservation standpoint comprises bio-based resins such as PLA and PHA isocyanate (e.g. PMDI) and polyol soybean oil. As a bio-based alternative to soybean oil, the following bio-based oils may be utilized in the resin material: corn, canola (a.k.a. rape seed), sunflower, oil palm, coconut, cotton, safflower, peanut, olive, and/or any other similar bio-based oil.

Although suitable virgin plastic resins as described above may be used as a plastic constituent of the bio-plastic composite material, suitable recycled plastic materials are preferred for environmental and economical reasons. Recycled thermoplastics such as polypropylene and various grades of polyethylene have been successfully integrated with biological materials to form a bio-plastic composites by methods of the present invention. Other suitable thermoplastic recycles or mixture thereof may be used to make various bio-plastic composite materials.

Odor Controlling Agents

One preferred embodiment provides for odor controlled bio-plastic composites. When a mixture containing a biological material is processed at an elevated temperature, the biological material can generates an unpleasant bio-odor. Particularly, bio-plastic composites made with hydrolyzed biological materials have a strong malodor. Bio-odors can occur for numerous reasons including decay, processing, burning and/or other reasons. Embodiments herein provide a method of counter acting the bio-odor by adding odor controlling agents. The odor controlling agents include odor reducing agents such as activated carbon and/or steam activated anthracite. These odor reducing agents adsorb malodorous molecules in the processed biological material, thereby reducing the malodor. Other examples of odor reducing agents include, but not limited to, baking soda and molecular sieve materials such as zeolite or silica gel may also be used. If addition of fragrance is desired, suitable fragrances may be selected from those compiled by the U.S. Food and Drug Administration in Title 21 of the Code of Federal Regulations, Sections 172.510 and 172.515, incorporated by reference herein. A fragrance oil used in embodiments of the invention may include, for example, fragrance components seletect from benzaldehydes, phenols, cinnamic aldehydes and esters, octadienes, dienes, cyclohexadienes, and terpenes.

These odor reducing agents may be in a fine powder form and may be integrated at different stages of making bio-plastic composite materials. For example, the odor reducing agent may be integrated with a plastic material and a biological material in an extrusion process where bio-plastic composite pellets are produced for further manufacturing processes such as injection molding or injection blow molding. Or the odor reducing agent may be mixed with a plastic material and a classified biological material prepared according to the method described in later this section, and fed into bio-plastic composite manufacturing processes such as injection molding or sheet extrusion processes.

Another odor controlling method is fragrances and/or odor neutralizers such as Odourfoyl products marketed by Belmay Fragrances Ltd. An odor is made up of airborne molecules that interact with receptor cells of a human nose. The fragrances and odor neutralizers works by altering the chemistry of the molecule so that the receptor cells no longer recognize the molecule as a malodor. These products interact with malodorous molecules and distort the molecules to make them undetectable as malodors to receptors in the nose. In the process of working with the olfactory, the malodor is effectively eliminated and replaced by fragrances or by an odor-neutral effect. Some examples of such fragrance compounds include “fresh and clean,” “citrus,” “cedar,” “oak,” etc. When bio-plastic composites are used to replace wood, for example, cedar or oak fragrances or other wood fragrances can have benefit.

The fragrances and/or odor neutralizers may be integrated into bio-plastic composite materials at different stages. However, it is desirable to minimize flashing of these materials, particularly during processes involving elevated temperatures. One preferred method of integrating fragrances and/or odor neutralizers into a bio-plastic composite material is by adding a suitable polymeric material. One suitable polymeric material is polyethylene vinyl acetate (EVA) beads impregnated with the fragrances and/or odor neutralizers. EVA is a copolymer of ethylene and vinyl acetate. The EVA has no odor by its nature, however, it can adsorb or otherwise be permeated a fragrance, an odor neutralizer, a corn oil, and/or color additives. EVA approaches elastomeric materials in softness and flexibility, yet can be processed like thermoplastics. Such characteristics of EVA allow the additives to be impregnated in EVA resin.

Other suitable polymeric materials share the beneficial properties of EVA and may be substituted for use in embodiments of the invention. These include, for example, but are not limited to ethyl vinyl alcohol, high density polyethylene, low density polyethylene, polystyrene, acrylic polymers, polycarbonates, cellulose acetate, cellulose nitrate, nylons, and mixtures and copolymers of the foregoing. Exemplary cellulose compositions are reported, for example, in U.S. Pat. No. 2,169,055, to Overshiner, et al. Cellulose compounds may be produced in solution with an organic solvent and a fragrance and/or odor neutralizer. Suitable solvents include, for example, acetone and 1,4 diethylene oxide.

Plasticizers may also be added to polymeric materials that are used in embodiments of the invention. These may include, for example, diethyl phthalate and tri-acetic acid ester of glycerin.

In further embodiments of the invention, fragrances and/or odor neutralizers include one or more hindered amines. The hindered amines useful in the instant invention are well known in the art and are described in detail in U.S. Pat. No. 6,221,115, the relevant parts of which are incorporated herein by reference. Examples of the hindered amines are: 1-(2-hydroxy-2-methylpropoxy)-4-octadecanoyloxy-2,2,6,6-tetramethylpiperi-dine; 1-(2-hydroxy-2-methylpropoxy)-4-hydroxy-2,2,6,6-tetramethylpiperidin-e; bis(1-octyloxy-2,2,6,6-tetramethylpiperidin-4-yl) sebacate; bis(1-cyclohexyloxy-2,2,6,6-tetramethylpiperidin-4-yl) sebacate; 1-cyclohexyloxy-2,2,6,6-tetramethyl-4-octadecylaminopiperidine; 2,4-bis [(1-cyclohexyloxy-2,2,6,6-tetramethylpiperidin-4-yl)butylamino]-6-(2-hydroxyethylamino-s-triazine; bis(1-cyclohexyloxy-2,2,6,6-tetramethylpiperidin-4-yl) adipate; 1-(2-hydroxy-2-methylpropoxy)-4-oxo-2,2,6,6-tetramethylpiperidine; bis(1-(2-hydroxy-2-methylpropoxy)-2,2,6,6-tetramethylpiperidin-4-yl) sebacate; bis(1-(2-hydroxy-2-methylpropoxy)-2,2,6,6-tetramethylpiperidin-4-yl) adipate; bis(l-(2-hydroxy-2-methylpropoxy)-2,2,6,6-tetramethylpiperidin-4-yl) succinate; bis(1-(2-hydroxy-2methylpropoxy)-2,2,6,6-tetramethylpiperidin-4-yl) glutarate; and 2,4-bis {N-[1-(2-hydroxy-2-methylpropoxy)-2,2,6,6-tetramethylpiperidin-4-y-1]-N-butylamino}-6-(2-hydroxyethylamino)-s-triazine) 1-methoxy-4-hydroxy-2,2,6,6-tetramethylpiperidine; 1-methoxy-4-hydroxy-2,2,6,6-tetramethylpiperidine; 1-octyloxy-4-hydroxy-2,2,6,6-tetramethylpiperidine; 1-cyclohexyloxy-4-hydroxy-2,2,6,6-tetramethylpiperidine; 1-methoxy-4-oxo-2,2,6,6-tetramethylpiperidine; 1-octyloxy-4-oxo-2,2,6,6-tetramethylpiperidine; and 1-cyclohexyloxy-4-oxo-2,2,6,6-tetramethylpiperidine, or a mixture thereof.

In yet further embodiments of the invention, fragrances and/or odor neutralizers include one or more antioxidants. Antioxidants used in embodiments of the invention may be, for example, tertiary butylhydroquinone, n-octadecyl 3,5-di-tert-butyl-4-hydroxyhydrocinnamate, butylated hydroxyanisole, phenol bisphosphite, butylated hydroxytoluene, and phosphite compounds. An effective amount of antioxidant in the instant composition is 0.015% to 2.5% by weight of the EVA or other polymer, preferably 0.1 to 0.75% by weight and most preferably 0.2 to 0.5% by weight. In preferred embodiments of the invention, high concentrations of antioxidants are mixed with fragrance priori to addition of the fragrance/antioxidant mixture to any other components of the mixture.

Still further embodiments of the invention contemplate inclusion of the fragrance and/or odor neutralizer in a diluent. A diluent is organic, for example: triethyl citrate; di-isopropropyl adipate; di-octyl adipate; isopropyl myristate; isopropyl palmitate; butyl stearate; benzyl alcohol; benzyl benzoate; and diethyl pthalate. The quantity of diluent preferred is the quantity necessary for dissolving the fragrance or the antioxidant.

In one preferred embodiment, a selected fragrance and/or an odor neutralizer (with or without the other additives reported above) is embedded in and/or adsorbed on the polymer. This tends to prevent these products from flashing off/burning off during plastic manufacturing processes involving heating. The fragrance and odor neutralizer survive the heated process, protected by the surrounding polymer molecules, then distributed throughout the bio-plastic composite material to counter act malodorous molecules in the processed biological material. One example of such polymer beads adsorb up to 65% by weight odor neutralizer and 35% by weight fragrance. Methods of integrating fragrances/odor neutralizers impregnated polymer beads into bio-plastic composites are described in later this section. It should be noted that although “beads” is used generally in discussion of the polymer, no particular shape is required.

In one embodiment, the beads include fragrance and/or odor neutralizer are prepared by first mixing the fragrance and/or odor neutralizer with at least enough diluent sufficient to dissolve the fragrance and/or odor neutralizer. Other additives are added to the resulting solution, with additional diluent added as desired to maintain dissolution of the added substances. The mixture is then mixed with polymer beads (for adsorbtion) or with molten polymer beads (for adsorbtion and inclusion) to create the fragrances and/or odor neutralizer-bearing beads. Further information regarding creation of a fragrance/antioxidant/diluent mixture may be found in U.S. Pat. No. 7,220,288, which is incorporated by reference as if fully rewritten herein.

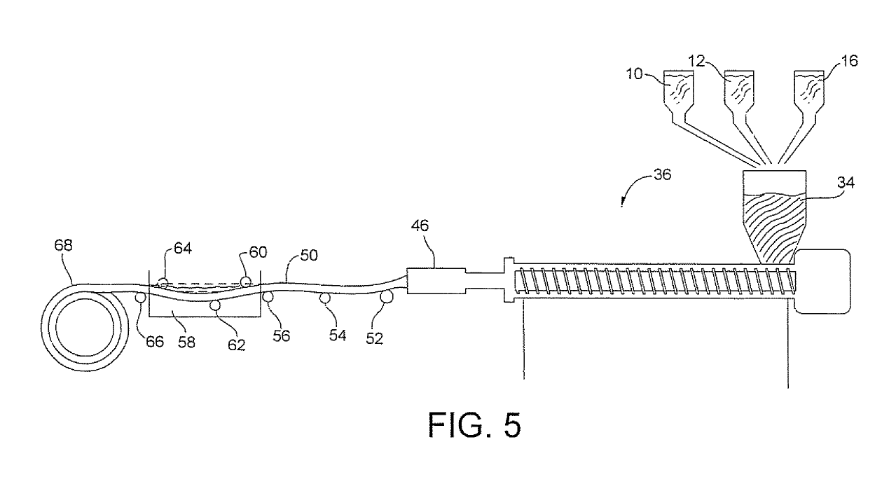

Alternatively, fragrances and/or odor neutralizers may be applied after a bio-plastic composite material is formed. As shown in FIG. 5, in one embodiment, a bio-plastic composite sheet stock exiting an extruder die is directed into a dip tank with a liquid fragrance or odor neutralizer. The fragrance or odor neutralizer may be compounded with a liquid coating material such as polyurethane to encapsulate the fragrance and/or odor neutralizer in the outer coating layer of the bio-plastic composite material. Yet, in another embodiment, the fragrance and/or odor neutralizer may be spray coated on to bio-plastic composite products such as injection molded pieces. As such the odor control agent may not be integrated in plastic and be in fluid or powder form.

Other Additives

Various other additives such as corn oil, color additives, plasticizer, etc. may be added in different embodiments depending on desired characteristics of a particular bio-plastic composite material. In one preferred embodiment, recycled tire is cryogenically pulverized with the biological materials and added to the bio-plastic composite material as a filler alternative.

In another embodiment, corn oil may be added to minimize burn coloring of bio-plastic composites. The corn oil in this embodiment is crude-degummed corn oil. This is oil that has been recovered by pressing and extracting the germ portion of the corn kernel. It is then filtered and degummed by removing the majority of the phospholipids. Other grades of corn oil such as RB corn oil, RBD corn oil, and RBDW corn oil made be used in other embodiments where different degrees of clarity is desired.

RB corn oil is refined to remove the majority of free fatty acids. It is then bleached to remove a large portion of the color bodies. RBD corn oil is deodorized to remove even more color bodies and odor compounds. RBDW corn oil is further processed to remove even more color bodies by removing waxes.

### Methods of Preparing Bio-Plastic Composite Material Constituents

When a biological material is used in a composite, there may be a defined drop in strength characteristics of the composite. Part of problem with using biological materials is inconsistency of particular bio products from location to location and/or processed biological byproducts from different manufacturing facilities. For example, in the case of ethanol production, there are vast differences in byproduct DDGs from facility to facility and between production runs. A consistency in particle size of the biological material in a composite controls or maintains uniformity of strength and stiffness characteristics of the composite. In general, finer and drier biological material results in a bio-plastic composite with superior characteristics in both thermoplastic and thermoset applications. Therefore, one aspect of the present invention provides for different methods of preparing biological material.

Classification

One preferred embodiment of processing biological material is classification. Different biological materials may be processed differently according to the nature of the biological material prior to entering a classification process. For example, DDGs may simply be fed into a classification system, without any other additional preparation steps, where DDGs are fluidized and transported with an upward stream of air into a sieve with a selected mesh size according to desired characteristics of the bio-plastic composite material. Wet mills may be dried using any conventional drying process such as a batch drying system to a desired moisture content before entering the classification process. Other biological materials may require additional particle size reduction process step.

In one embodiment, the particle size reduction of the biological material process involves hammer milling. In hammer milling, particles are reduced in size by rapidly moving surfaces. An example of such a device is rapidly rotating hammers that strike particles repeatedly until the particles are reduced in size and pass through a nearby screen. Hammer milling is typically done at ambient conditions to produce particles of 30-200 mesh through a 75 to 500 μm sieve and classified by adding the material to the screen and then shaking the screen to produce an “overs” and an “unders.” The “overs” are the particles that remain on the screen and the “unders” are the particles that pass through the screen. In a continuous process, the particles are continuously added to a screen and the “overs” continuously removed so as to avoid blinding or plugging the screen.

In another embodiment, a biological material may first be processed through a drying step such as batch drying before entering the milling process, depending on the moisture content of the biological material. Alternatively, the milled biological material may be classified to a desired size range first, then dried to a target moisture content.

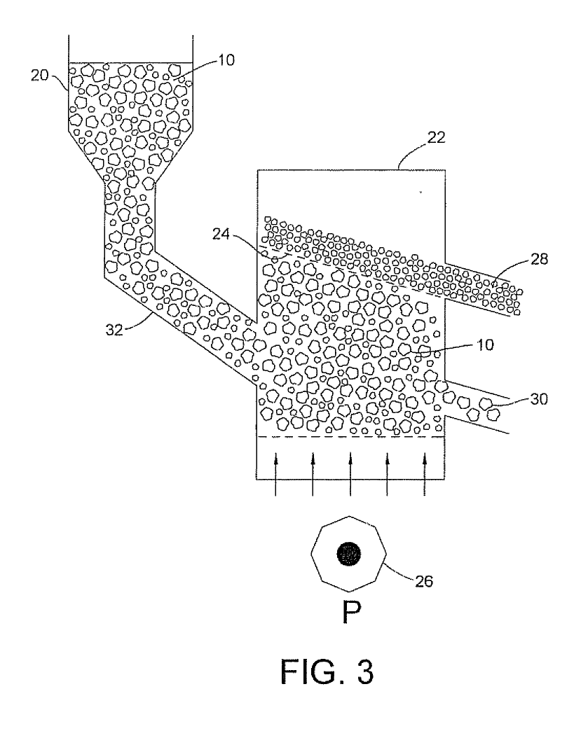

The classification process may involve a single sieving step or multiple sieving steps. FIG. 3 illustrates one embodiment of the classification process. In this embodiment biological material 10 is fed into a hopper 20. The feeding of the biological material into the hopper 10 may be continuous or a batch operation. The biological material 10 is transferred to an air sieve classification system 22 through a conduit 32 connecting the hopper 10 and the classification system 22. A pressurized air supply 26 provide an upward stream of air from the bottom of the classification system 22. The upward stream of air fluidizes and pushes the biological material 10 toward a sieve 24 with a mesh size selected according to the nature of the biological material and a desired range of particulate sizes. The biological material is then separated into classified particulates 28 and coarse particulates 30.

In another preferred embodiment, the air sieving classification may involve multiple sieving stages where different mesh size sieves are used. In this embodiment, the biological material is first classified to separate fiber parts from non-fiber parts, then the fiber parts are further classified into desired particle size ranges.

In one embodiment the classification is done using centrifugal forces. A particle separation equipment utilized in this embodiment operates by applying opposing air flows and centrifugal forces. By balancing the two forces, smaller and larger particles can be separated. Good separation is usually obtainable down to 2 μm. Depending on the size of the screen set or the classifier rates, classification can be as low as one pound per hour to as high as thousands of pounds per hour.

In some embodiments, a size reduction process and a classification process is combined into a continuous process. For example, the biological materials may be hammer milled then continuously fed into the one of above described classification system for continuous separation. The classified biological material may be supplied to manufacturers, in its powder form, to be used in their processes such as injection molding process, or it may be integrated with a plastic material and/or other additives into pellets for use in further manufacturing processes. The method of making pellets and methods of using the pellets will be discussed in later sections.

The classification method of the present invention controls or even may improve the strength properties of the bio-plastic composite material by providing a method to select particulates with a specific range of particle sizes. Additionally, in the case of some bio-plastic composites, the bio-particles must readily pass and not clog screens such as in plastic injection molding. Generally, finer and drier the biological material particulates produce bio-plastic composites with better properties. Composite properties enhanced by use of the classified biological fiber include flexural modulus, flexural strength, tensile modulus, tensile strength, tensile elongation, and Charpy impact. Table 1 shows test results comparing a bio-plastic composite material comprising a classified biological material and polypropylene against virgin polypropylene and a glass fiber filled polypropylene composite.

TABLE 1

| | Classified ground | | | | |
|---|---|---|---|---|---|
| | corn cob material, | | | Glass | |
| | 10% by weight, | | | fiber filled | |
| | filled polypropylene | | Virgin | polypropylene | |
| Property | composite | | polypropylene | composite | |
| flexural modulus | >6.5 | MPa | ~1.300 MPa | 5.8 | MPa |
| flexural strength | >100 | MPa | ~42 MPa | 105 | MPa |
| flexural | ~3.0% | | ~7% | — | |
| elongation | | | | | |
| tensile modulus | >6.1 | MPa | | 5.6 | MPa |
| tensile strength | >65 | MPa | | 70 | MPa |
| tensile | ~2.5% | | | 2.2% | |
| elongation | | | | | |
| Charpy impact | >20 | kJ/m² | | 23 | kJ/m² |

### Hydrolysis

In one preferred embodiment, the biological material is hydrolyzed before it is integrated with other composite constituents. One effective method of hydrolyzing the biological material is LignoTech. When a biological material is hydrolyzed using LignoTech, the process starts with comminuting the biological material to a size so the material can be effectively gunned in hydrothermal pressure vessels. Preferably, the particle size of the biological material entering the hydrothermal pressure vessels should fall within the range of length up to 40 mm, width up to 6 mm and a height of up to 6 mm. The thickness of the biological material no greater than 5 mm is preferred for best results. However, biological material particulates with greater sizes than these preferred particle size ranges may also be processed effectively.

The comminuted biological material is then dried, preferably in a cyclonic drier at an appropriate temperature according to the nature of the material. The temperature of the drying system is selected to prevent any damages to the biological material due to a high temperature. When the biological material is dried in moving air, the air velocity is regulated along with the temperature of the air to ensure adequate drying of the material, preferably to a moisture content between 11% to 25%, although a higher moisture content may also work for some applications. The best results have been obtained with the dried material with around 16% moisture content.

The dried material is then packed into a hydrothermal reactor for thermal hydrolysis. The reactor is injected with dry or up to 5° C. superheated steam at a pressure preferably below 65 bar, or preferably between 32 to 45 bar. The pressure and temperature are selected to ensure the material does not burn and or unduly deteriorate in its physical characteristics. Optimal conditions of the hydrolysis may be obtained with 100% dry steam. For some biological materials, the steam may be slightly superheated to accelerate the initial chemical reaction and reduce the condensation in the reactor vessel while pressure is being built up to the required amount. The hydrolysis process usually takes between 30 to 100 seconds, but may take up to ten minutes. Higher pressure, temperature, or longer time may be required depending on the nature of the biological material.

After completion of the hydrolysis process, the vessel is decompressed. The decompression step usually takes less than 2 seconds. The processed material is then cooled down to prevent further chemical reaction. The material is then dried at temperature between 55° C. and 90° C., preferably, below 75° C. The material is dried until a moisture content under 10% is obtained, preferably, under 3%.

The hydrolyzed biological materials have characteristics to replace plastic materials in bio-plastic composite materials. In one embodiment, a bio-plastic composite may consist of up to 99% by weight of the hydrolyzed biological material. Preferably, a more substantial amount of plastic material is incorporated to afford different plastic manufacturing processes such as injection molding for example.

Unfortunately, the biological materials generate strong unpleasant malodor when they are hydrolyzed. This unpleasant bio-odor carries throughout subsequent manufacturing processes, and remains in final bio-plastic composite products. Thus, the bio-plastic composite products made from the hydrolyzed biological materials are not readily marketable due to the strong malodor. Therefore, one aspect of the present invention integrates the odor controlling agents, as described above, to reduce or eliminate the malodor.

In one embodiment, the odor reducing agents such as activated carbon, fragrances, and/or odor neutralizers may be mixed with a biological material in the pressurized vessel. Alternatively, the fragrance/odor neutralizer impregnated EVA beads may be added to the vessel with the biological material. In such embodiments, the odor controlling agents will interact with malodorous molecules of hydrolyzed biological material in the vessel.

In other embodiments, the fragrance/odor neutralizer impregnated EVA beads are mixed with a hydrolyzed biological material and a plastic material before the mixture is fed into a bio-plastic composite manufacturing process. For example, in one embodiment, a mixture comprising 70% hydrolyzed DDG, 28% recycled polypropylene, and 2% fragrance and odor neutralizer filled EVA beads is injection molded to produce a bio-plastic composite product.

Cryogenic Grinding

According to another aspect of the present invention, the biological material is cryogenically pulverized to produce a powder biological material. The cryogenic grinding may be used for any biological material discussed above, however, it is particularly effective for tougher biological materials such as hay, switchgrass, kenaf, etc. Moreover, the cryogenic grinding process make it possible to pulverize the biological material together with recycled tires. Utilization of the recycled tires are very limited today; they are mostly deposited in landfills, thus available readily for very low cost. Moreover, high temperature plastics such as polyamides may be recycled by cryogenic grinding to be used as a filler alternative in bio-plastic composite materials.

In one such embodiment, biological materials and recycled tires are first chopped into small enough pieces for cryogenic grinding. Then, a mixture of chopped biological materials and recycled tires is frozen using a cryogen such as a liquid nitrogen at around −320° F. The mixture is then shattered like a glass thrown against the wall and put through screens according to a desired particle size of the carbon black powder. The pulverized biological materials or the mixture of biological material and recycled tires may be integrated with any suitable plastic material to make a bio-plastic composite material. The pulverized materials may also be used in conventional composites as a filler replacing products like talc.

### Methods of Making Bio-Plastic Composite Materials

Pelletizing

The biological material processed by one of above discussed methods (classification, hydrolysis, or cryogenic pulverization) may be integrated with a suitable plastic material and pelletized for temporary storage, transport, and/or further manufacturing such as injection molding, blow molding, and/or thermoforming. For example, a selected processed biological material may be integrated with a thermoplastic material then extruded. The thermoplastic material is preferably ground recycled thermoplastics, but it could also be other suitable virgin plastic resins such as polypropylene, polyethylene, polystyrene, polyester, PVC, ABS, etc.

In the pelletizing process, a selected processed biological material and a suitable thermoplastic material are placed in an actuation tank for mixing, then gravity fed from a top mounted hopper into an extruder. The fragrance and/or odor neutralizer impregnated EVA beads may be added to the mixture. Although the preferred method of adding fragrances and/or odor neutralizers is by impregnating the products first in EVA beads, the fragrances and/or odor neutralizers may be added by themselves for some embodiments. Alternatively, odor reducing agents such as activated carbon may be mixed with the biological and plastic materials.

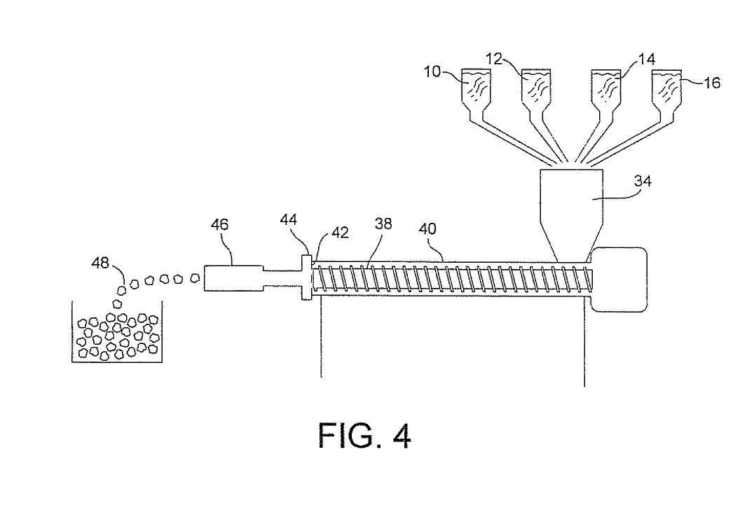

FIG. 4 shows one embodiment of the pelletizing process. In this embodiment, a processed biological material 10, a plastic material, 12, an odor controlling agent 14, and additives selected for desire bio-composite properties are fed into a hopper 34 for mixing. Each constituent is fed from an individual hopper to allow individualized control of the amount fed into the hopper 34. The mixed constituents in the hopper 34 is then gravity fed into an extruder 36.

In the extruder 36, a rotating screw 38 forces the mixture forward in the extruder barrel 40 which is heated to a melting temperature of the plastic material, usually around 400 F. Such extruder 36 is preferably equipped with multiple independently controlled heating zones to enable gradual heating of the mixture as it moves through the barrel 40. Often, the extruder 36 also has cooling devices to counteract a rise in temperature from excessive pressure in the barrel 40. This lowers a risk of overheating which may cause degradation in the polymer and the biological material.

The molten mixture in the extruder barrel 40 is forced through a screen pack 42 located near an outlet of the extruder where any contaminants in the molten mixture is removed. The screen pack 42 is reinforced by a breaker plate 44 which together with the screen pack 42 provide a back pressure in the barrel necessary for uniform melting and mixing of the materials in the barrel. Once screened, the molten mixture is forced out of the extruder, into a die 46 which forms the mixture into final shapes such as pellets/beads 48 in different sizes.

The pellets/beads may be used in subsequent bio-plastic composite manufacturing processes described below. These pellets/beads exhibit composite strength characteristics resulting from even distribution of the biological fiber material throughout the pellets/beads. Therefore, the pellets may also be used in a cement laying process. When cement is mixed with water and other components, the water reacts with cement and solidifies into a stone-like material, concrete. The concrete is used in various construction applications, such as pavements, architectural structures, foundations, roads, overpasses, brick/block walls, etc.

Traditionally, both fine and coarse aggregates such as sand, natural gravel, and crushed stones are utilized to make up the bulk of the concrete mixture. A typical batch of concrete may include 1 part cement, 2 parts dry sand, 3 parts dry stone, and ½ part water (parts in terms of weight). The bio-plastic composite pellets may replace these aggregate components of the concrete. The bio-plastic composite pellets can increase strength characteristics and/or lower the density of the cement.

Extrusion

The same type of extruder and the method used in above described pelletizing process may also be used to produce bio-plastic composite sheet stock simply by exchanging the die.

Injection Molding

The bio-plastic composite pellets may be used in an injection molding process, and molded into desired shapes and sizes. In the injection molding process, the bio-plastic composite pellets are fed from a hopper into a molding machine where a reciprocating screw carries the pellets through a heated barrel. Where the pellets were extruded without fragrances and/or odor neutralizer, fragrance and/or odor neutralizer filled EVA beads may be added to the hopper. In the barrel, the heat from the heating modules and shear generated by the flights of the screw melts the plastic. Then the screw conveys the molten mixture toward the front of the barrel as it melts and mixes the mixture to uniformity.

The screw retracts as molten bio-plastic mixture accumulates in the front of the barrel, then when the enough molten mixture accumulates to fill the mold, the screw is pushed forward hydraulically. This forces the molten mixture through the machine nozzle and into the closed mold. In the mold, the molten mixture flows through channels called runner and passes into part cavities through gates. Water or another fluid circulating through a cooling system in the mold extracts heat. The mixture is held at high pressure until it solidifies, or freezes off, at the gates. After parts have cooled and solidified enough to be handled, the mold is opened and the parts are removed.

Alternatively, a processed biological material mixed with a suitable plastic material and other additives may be fed directly into the injection molding hopper without being pelletized first.

In one preferred embodiment, corn cobs are hammer milled and classified to particle size under 400 microns by the air sieving method described above. The ground material is dried in a batch drier at around 100° C. (212° F.) to obtain the material with moisture content less than 0.3% by weight. The dried corn cob material can be stored in a moisture proof container or mixed with ground recycled polyethylene for immediate injection molding process. This embodiment may constitute 20-30% by weight of classified corn cob material and 1-3% by weight of fragrance filled EVA integrated with recycled polyethylene particulates.

In this embodiment, it is important to minimize the time bio-plastic material mixture is exposed to high temperatures. It is important to match the part or shot size to the barrel volume. An excessively large barrel volume/part volume ratio will expose the materials to for unnecessarily long times and result in smoke generation and dark or charred parts. It is also advisable to purge these materials from the molding system after molding is complete to avoid time induced charring. This bio-plastic composite material should be molded at as low a temperature as possible to avoid charring and smoke generation, preferably under 392° F. (200° C.). All barrel and nozzle temperatures is recommended to be set below this temperature.

Injection Blow Molding

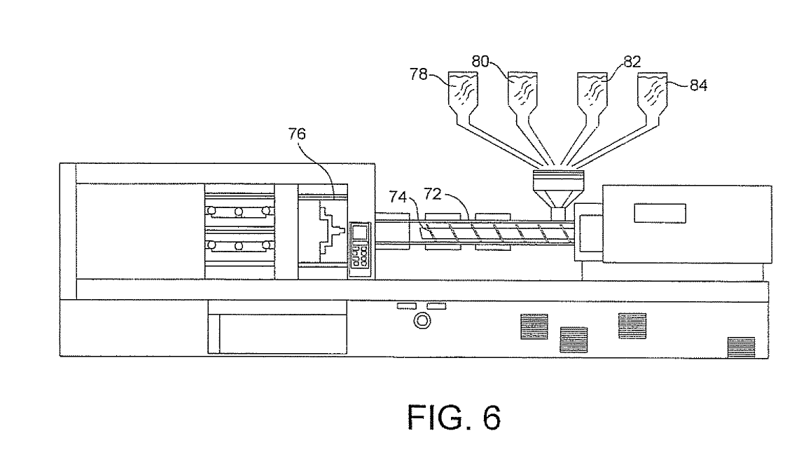

The bio-plastic composite pellets may be used in an injection blow molding (IBM) process to produce hollow objects. FIG. 6 illustrates an IBM machine. This IBM machine 70 has an extruder barrel 72 and screw assembly 74 which melts the pellets. Where the bio-plastic composite pellets without fragrance and/or odor neutralizer are fed, the fragrance and/or odor neutralizer filled EVA beads may be fed with the pellets. The molten mixture is fed into a manifold where it is injected through nozzles into a hollow, heated preform mold 76. The preform mold forms the external shape and is clamped around a mandrel or core rod which forms the internal shape of the preform. The preform mold opens and the core rod is rotated and clamped into the hollow, chilled blow mold. The core rod opens and allows compressed air into the preform which inflates it to the finished shape.

Alternatively, a processed biological material mixed with a suitable plastic material and other additives may be fed directly into the extrusion barrel of the injection blow molding machine without being pelletized first. Multiple individual hoppers 78-84 allow controlled feeding of each constituent of a bio-plastic composite into the extruder barrel 74 of the IBM machine.

Coextrusion

Coextrusion refers to the extrusion of multiple layers of materials simultaneously. In coextrusion, two or more extruders are utilized to melt and deliver a steady volumetric throughput of different molten bio-plastic composite materials to a single extrusion head which combines the materials in a desired shape. The thickness of each layer is controlled by the relative speeds and sizes of the individual extruders delivering the materials.

There are variety of reasons for use of the coextrusion process over the single layer extrusion. One example is in the fencing industry where thin outer layers of bio-plastic composite material with expensive weather resistant additives are extruded on a thicker layer of bio-plastic composite material designed for enhanced impact resistance and structural performance.

Thermoforming

The extrude bio-plastic composite sheet stock may be further processed by thermoforming. In the thermoforming process, a bio-plastic sheet stock is heated till soft, and formed on a mold into a new shape. When vacuum is used the process is often described as vacuum forming. Thermoforming can go from line bended pieces, such as displays, to complex shapes like computer housings. With help of various thermoforming technology such as inserts, undercuts, and divided molds, many thermoformed pieces are comparable to injection molded parts.

### Odor Controlled Bio-Plastic Composites

As described above odor controlling agents may be integrated at various different stages of process of making an odor controlled bio-plastic composites. Moreover, odor controlled composites may be made by using different combinations of methods of preparing constituents and methods of making bio-plastic composites as described. For example, any one of methods of processing biological material, i.e. hydrolysis, classification, and cryogenic grinding may be combined with any one of manufacturing methods, i.e. extrusion, injection molding, injection blow molding, coextrusion, and thermoforming to make a desired bio-plastic composite.

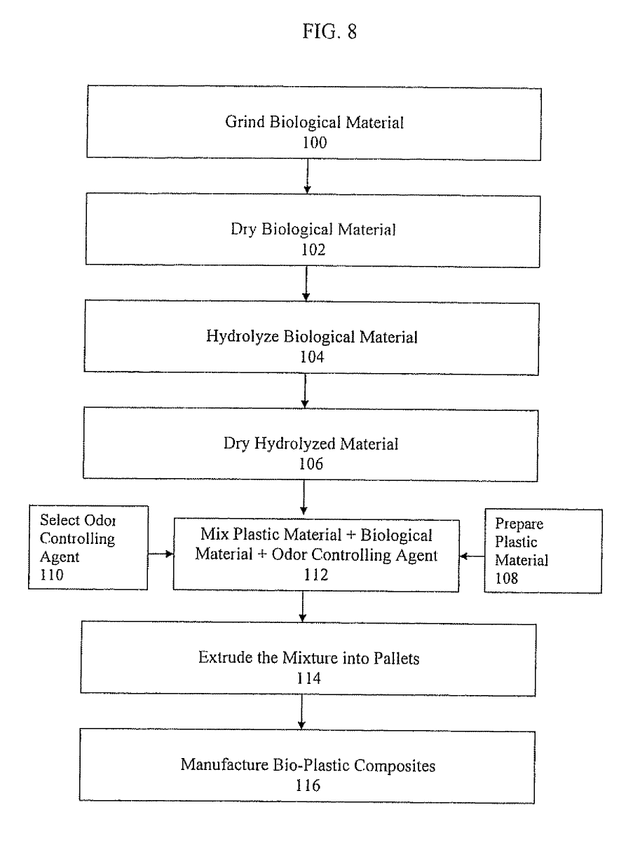

FIG. 8 shows a process flow diagram of one embodiment of making odor controlled bio-plastic composites. In this embodiment, the process starts with a step of grinding biological material 100 using a particle size reduction method such as hammer milling as described previously. Alternatively, the ground biological material may be DDG which is already in particulate form not requiring additional grinding step. Next, the biological material particulate is dried 102 appropriately for a hydrolysis process. Then the biological material is hydrolyzed 104 in a pressurized hydrothermal vessel. The hydrolyzed material is then dried again 106 to a desired moisture content. In parallel with these biological material preparation steps, a plastic material is also prepared 108 either by obtaining a suitable virgin polymeric resin or grinding recycled thermoplastic material. A proper odor controlling agent such as fragrance and/or odor neutralizer impregnated EVA beads is also selected 110. The dried hydrolyzed material is then mixed with the prepared plastic material and the selected odor controlling agent 112. The mixture is then extruded in an extruder into pellets 114. The odor controlled bio-plastic pellets are then used as input material in various manufacturing processes 116 such as injection molding, injection blow molding, sheet stock extrusion, or coextrution.

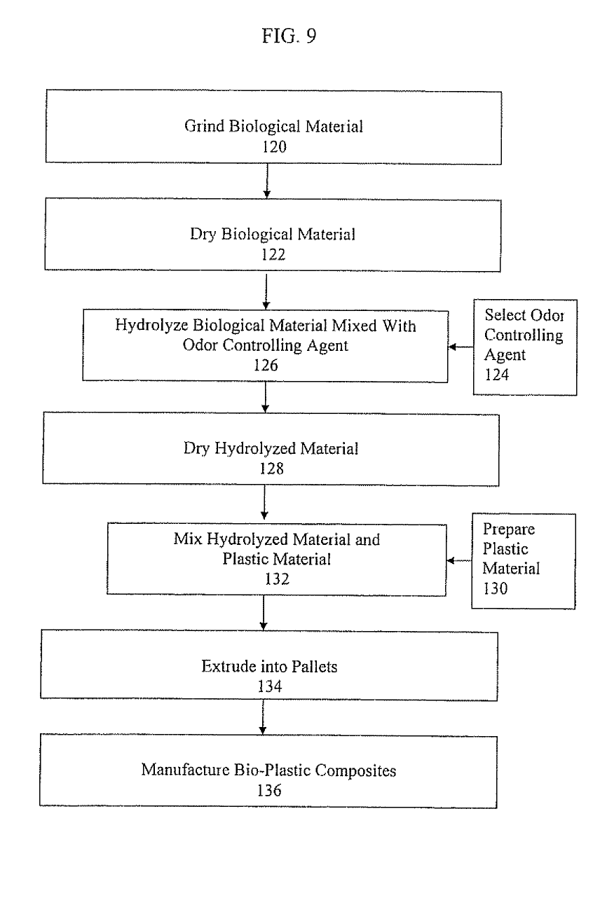

FIG. 9 shows a process flow diagram of another embodiment. This embodiment comprises mostly same process steps as the embodiment illustrated in FIG. 8, except the odor controlling agent in this embodiment is added into the pressurized hydrothermal vessel 126. Therefore, in this embodiment a bio-odor generated during the hydrolysis process is already eliminated or masked in the pressurized hydrothermal vessel. Additional odor controlling agent may be added at a later process stage, if needed.

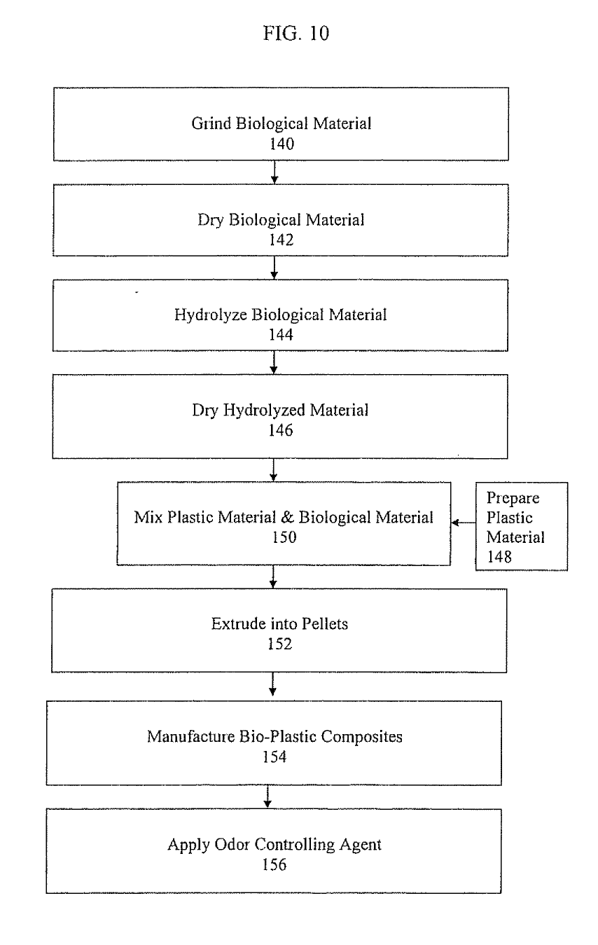

FIG. 10 shows a process flow diagram of yet another embodiment. This embodiment comprises mostly same process steps as the embodiment illustrated in FIG. 9, except the odor controlling agent is not mixed with the biological material and the plastic material in the extruder 150. Thus, the bio-plastic composite pellets 152 in this embodiment have a bio-odor generated from hydrolyzing the biological material. This bio-odor remains in the bio-plastic composites produced by various manufacturing processes. Therefore, this embodiment has an additional process step of applying an odor controlling agent 156 to the manufactured bio-plastic composites to eliminate or mask the bio-odor. In such post manufacturing application of odor controlling agents, bio-plastic composite products may be sprayed or dipped in a fragrance and/or odor neutralizer. In some applications, fragrances and/or odor neutralizer are mixed with a liquid coating material to coat bio-plastic composite products.

FIG. 5 illustrates one embodiment of applying a fragrance or an odor neutralizer by dipping a bio-plastic composite sheet stock in a pan containing the fragrance or the odor neutralizer. In this embodiment a biological material 10, a plastic material 12 and/or selected additives 16 are mixed in a hopper 34 and gravity fed into an extruder 36 where the mixture is extruded into a bio-plastic composite sheet stock 50. The extrusion process here is same as the pelletizing extrusion process described above for the embodiment illustrated in FIG. 4, except the die 46 in this embodiment is configured to produce a sheet stock instead of pellets. The bio-plastic composite sheet stock 50 is directed by rollers 52, 54, and 56 into a dip tank 58 which contains a fragrance and/or an odor neutralizer in liquid form. The bio-plastic composite sheet stock 50 is guided by rollers 60, 62, and 64 in the dip tank 58 to ensure immersion of the sheet stock 50 in the fragrance and/or odor neutralizer. Then the sheet stock 50 is guided out of the dip tank 58 by a roller 66 into a roll of odor controlled bio-plastic composite sheet stock 68.

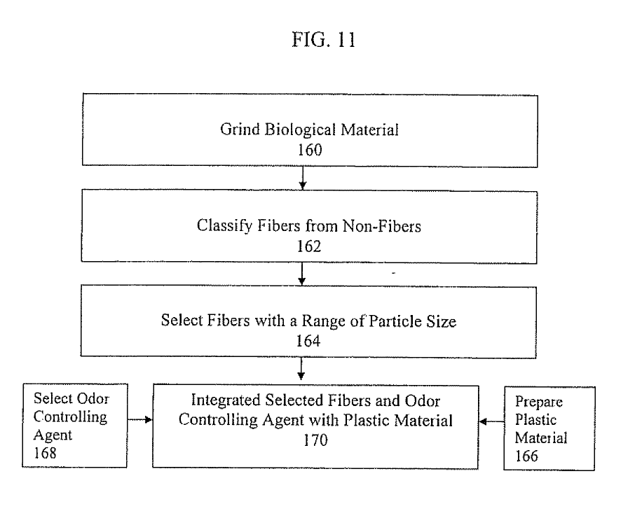

FIG. 11 illustrates a process flow diagram of another embodiment of making odor controlled bio-plastic composites. First step of this process is grinding biological material 160. Next, the ground biological material is classified, separating fiber material from non-fiber material 162, by using an air sieving method described previously. Then the fiber material is further classified to select a specific range of size particles 170. It is important to select appropriate size particles so the selected bio-particles can readily pass and not clog screens such as in injection molding. Concurrently, a plastic material is prepared 166, and a proper odor controlling agent is selected 168. Finally, selected bio-fiber particles and odor controlling agent is integrated with the plastic material 170 in one of manufacturing processes described above, i.e. extrusion, injection molding, injection blow molding, and/or coextrusion.

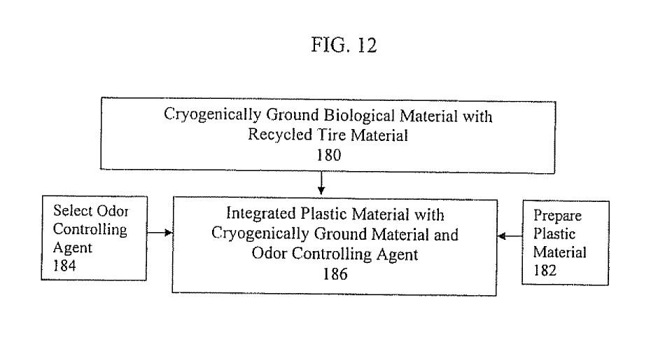

The process flow diagram of one embodiment shown in FIG. 12 starts with cryogenic grinding of biological material combined with recycled tire material 180. Similar to other embodiments, a plastic material is prepared 182 and an odor controlling agent is selected 184. Finally, the cryogenically ground material and the odor controlling agent is integrated with the plastic material 186.

### Benefits of Bio-Plastic Composite Materials

Besides the obvious environment conservation benefits, biological materials in the composite materials exhibit many other benefits. First, the biological material acts as a plastic extender. For example, different polymers used in injection molding process have different shrinkage rates, thus the polymer shrinkage has to be taken into consideration when calculating require amount of polymer for a production. However, the biological materials do not shrink under heat, thus acts as a polymer extender. In other words, when 50% biological material is mixed with 50% plastic material, 50% of the mixture will not shrink during the injection molding process, thus less overall shrinkage.

The biological material in bio-plastic composites also may reduce product density by replacing a higher density plastic material with a lower density biological material. The bio-plastic composites also can have enhanced strength characteristics when compared to products made only with plastic materials as shown in Table 2.

As discussed extensively above, one aspect of the present invention provide for methods of reducing or eliminating malodor from processing biological materials. Particularly, one embodiment eliminates a strong malodor of hydrolyzed biological material by adding fragrance and odor neutralizer impregnated EVA beads. This embodiment is particularly important since the hydrolysis process make it possible to dramatically increase amount of biological material in bio-plastic composites.

The cryogenically ground biological materials and recycled tires and/or recycled high temperature plastics such as polyamides in one embodiment may replace traditional filler materials such as talc and calcium carbonates. This bio-filler replacements are economically beneficial since otherwise non-recyclable waste materials are salvaged. Moreover, these bio-filler replacements are less abrasive than talc and calcium carbonates. Also, health hazards from talc and calcium carbonate dust are eliminated.

Examples of Bio-Plastic Composite Formulas with Classified Biological Materials

Various processed biological materials are integrated with different plastic materials to form bio-plastic composites and tested. Table 2 exhibits test results of various bio-plastic composites. As listed in the Table 2, maple wood flour, rice hulls, newsprint, kenaf fiber, cob fiber, hardwood cellulose in different amounts are integrated with polypropylene or polyethylene to form bio-plastic composites. These biological materials have been classified to particle size under 400 μm using a classification method as described above. The processed biological material is integrated in amounts of 25%, 40% or 50% by weight. For example, the formula PEMF40 consists essentially of 60% polyethylene and 40% processed cob fiber.

TABLE 2
Bio-Plastic Composite Performance Data - English Units

| A | B | C | D | E | F | G | H | I | J | K | L |
|---|---|---|---|---|---|---|---|---|---|---|---|
| PP | 0.90 | 126 | 2.82 | 108 | 3.42 | 0.62 | No Break | >32 | 0.05 | 202 | 4.9 |
| PPRH25 | 1.01 | 255 | 4.87 | 273 | 3.73 | 0.48 | 2.1 | >32 | 0.24 | 283 | 3.7 |
| PPCF25 | 1.01 | 290 | 5.76 | 275 | 6.00 | 0.73 | 10.4 | >31 | 0.33 | 312 | 2.6 |
| PPWF25 | 1.00 | 320 | 5.82 | 363 | 4.39 | 0.55 | 2.6 | >32 | 0.35 | 298 | 2.6 |
| PPNP25 | 1.01 | 323 | 5.80 | 367 | 4.60 | 0.56 | 4.0 | >31 | 0.39 | 298 | 3 |
| PPMF25 | 1.01 | 347 | 6.25 | 406 | 5.11 | 0.56 | 3.9 | >31 | 0.48 | 313 | 2.4 |
| PPKF25 | 1.00 | 371 | 6.36 | 437 | 4.93 | 0.50 | 3.1 | >32 | 0.39 | 303 | 2.4 |
| PPRH50 | 1.12 | 395 | 6.48 | 463 | 3.94 | 0.36 | 1.7 | 16 | 0.82 | 311 | 3.2 |
| PPNP50 | 1.11 | 555 | 8.65 | 643 | 5.95 | 0.57 | 3.0 | 13 | 1.2 | 315 | 2.0 |
| PPWF50 | 1.11 | 582 | 8.82 | 677 | 5.74 | 0.65 | 2.8 | 14 | 1.1 | 315 | 1.6 |
| PFKF50 | 1.12 | 762 | 10.13 | 908 | 6.87 | 0.65 | 2.9 | 20 | 1.4 | 319 | 1.5 |
| PPMF40 | 1.08 | 530 | 8.92 | 608 | 6.69 | .69 | 3.7 | 15 | 1.1 | 320 | 1.8 |
| PE | 0.95 | 77.7 | 1.64 | 62.7 | 2.34 | 0.56 | No Break | >20 | 0.01 | 147 | 7.7 |
| PERH25 | 1.04 | 157 | 2.91 | 203 | 2.47 | 0.42 | 2.0 | >33 | 0.26 | 224 | 5 |
| PEWF25 | 1.03 | 199 | 3.56 | 252 | 2.98 | 0.57 | 2.9 | >32 | 0.38 | 244 | 4.1 |
| PENP25 | 1.05 | 215 | 3.83 | 230 | 3.75 | 0.49 | 4.0 | 30 | 0.55 | 240 | 4.0 |
| PEMF25 | 1.05 | 229 | 4.05 | 281 | 3.87 | 0.75 | 3.0 | 25 | 0.9 | 256 | 3.2 |
| PEKF25 | 1.03 | 254 | 4.25 | 308 | 3.72 | 0.66 | 3.1 | >32 | 0.59 | 254 | 3.4 |
| PERH50 | 1.16 | 287 | 4.37 | 382 | 2.92 | 0.44 | 1.4 | 17 | 1.4 | 255 | 3.9 |
| PENP50 | 1.14 | 417 | 6.62 | 494 | 5.44 | 0.74 | 3.9 | 16 | 1.8 | 259 | 2.1 |
| PEWF50 | 1.14 | 447 | 6.19 | 581 | 4.31 | 0.70 | 2.8 | 17 | 1.5 | 255 | 2.1 |
| PEKF50 | 1.14 | 598 | 8.34 | 756 | 6.00 | 0.88 | 3.5 | 25 | 2.4 | 260 | 1.5 |
| PEMF40 | 1.05 | 407 | 6.49 | 490 | 521 | .94 | 3.4 | 19 | 1.4 | 261 | 2.4 |

WF = maple wood flour
RH = rice hulls
NP = newsprint
KF = kenaf fiber
MF = Cob fiber
CF = hardwood cellulose
A Formulation
B Specific Gravity (ASTM D792)
C Flexural Modulus (ASTM D790, 10³ psi); flexural modulus determined from slope between 20 and 40% of max strain.
D Flexural Strength (ASTM D790, 10³ psi)
E Tensile Modulus (ASTM D638, 10³ psi)
F Tensile Strength (ASTM D638, 10³ psi)
G Notched Izod Impact (ASTM D256, ft-lb in⁻¹)
H Un-notched Izod Impact (ASTM D256, ft-lb in⁻¹)
I Spiral Mold Flow (ASTM D3123, inches); spiral mold flow analysis conditions: nozzle and zones 1-3 set at 360° F., mold temp set at 80° F., RPM = 80, back pressure = 125 psi.
J 24 hr Water Absorption (Wt. %, ASTM D570); 24 hr moisture absorption determined per ASTM method except 2.5″ diameter × 1/16″ thick discs were used.
K Heat Deflection Temperature (ASTM D648, 66 psi, ° F.)
L Thermal Expansion Coefficient (AST D696 10⁻⁵° F.⁻¹)

Examples of Bio-Plastic Composite Formulas with Hydrolyzed Biological Materials

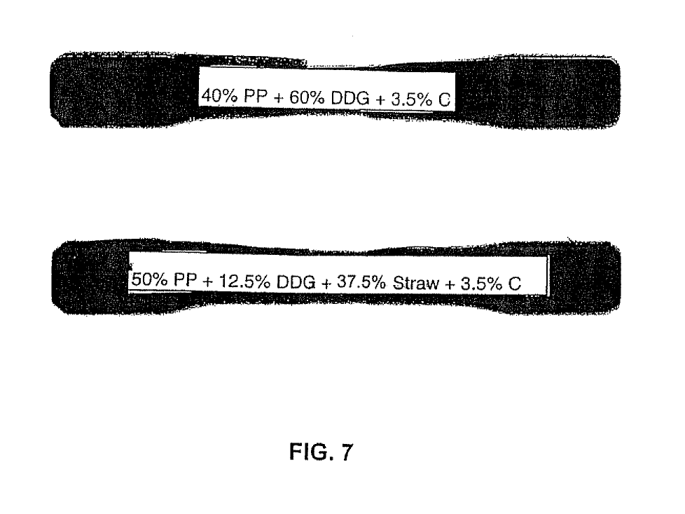

FIG. 7 shows various bio-plastic composite materials made from hydrolyzed biological material. These bio-plastic composites consist of between 40%-60% polyethylene or polypropylene, between 0%-50% DDG, between 0%-50% straw, and between 0%-3.5% filler. Bio-plastic composites with different biological materials exhibit different properties. For example, adding straw to DDG improves impact strength of the bio-plastic composite. Although not shown in these examples, other embodiments may include up to 99% of hydrolyzed biological material.

The use of the terms “a” and “an” and “the” and similar referents in the context of describing the invention (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. An odor controlled bio-plastic composite comprising:
a plastic material;
a processed biological material integrated with the plastic material, the biological material generating a bio-odor; and
an odor controlling agent integrated with the plastic material and arranged to counter act or mask the said bio-odor.

2. The odor controlled bio-plastic composite of claim 1 wherein said odor controlling agent is integrated in a polymeric material carrying at least one of a fragrance and an odor neutralizer, the polymeric material different than said plastic material, the polymeric material integrated with said plastic material.

3. The odor controlled bio-plastic composite of claim 2 wherein said polymeric material is EVA.

4. The odor controlled bio-plastic composite of claim 1 wherein said odor controlling agent is an odor reducing agent comprising at least one of activated carbon, activated anthracite, ziolite, silica gel, and baking soda.

5. The odor controlled bio-plastic composite of claim 1 wherein said processed biological material is fiber biological material in particle size appropriate to be processed in extrusion, injection molding, injection blow molding and coextrusion.

6. The odor controlled bio-plastic composite of claim 1 wherein said processed biological material is a hydrolyzed biological material prepared by a hydrolysis process.

7. The odor controlled bio-plastic composite of claim 1, said biological material comprising at least one byproduct of energy production generated from at least one of the group consisting of corn, soybean, flaxseed, switchgrass, rapeseed, miscanthus, hulls, stover, straw, bagasse from sugarcane and jatropha processed using a hydrolysis system.

8. The odor controlled bio-plastic composite of claim 1, said biological material comprising at least one of the group consisting of corn, soybean, wheat, barley, oats, sorghum(milo), sunflower, safflower, buckwheat, flax, peanut, rice, cnola, rye, millet, triticale, chickpeas, lentils, and filed peas.

9. The odor controlled bio-plastic composite of claim 1 wherein said processed biological material is a classified biological material prepared by a classification process.

10. The odor controlled bio-plastic composite of claim 1 wherein said processed biological material is a cryogenically ground biological material prepared by a cryogenic grinding process.

11. The odor controlled bio-plastic composite of claim 1, said plastic material comprising at least in part recycled thermoplastic material.

12. A method of making odor controlled bio-composite comprising steps of:
preparing a plastic material;
processing a biological material; and
forming an odor controlled bio-composite by integrating odor controlling agent with the biological material and the plastic material.

13. The method of making odor controlled bio-composite of claim 12 wherein the plastic material is prepared by at least one of thermoplastic material and reducing it to a size appropriate for integrating it with the biological material and the odor controlling agent.

14. The method of making odor controlled bio-composite of claim 12 wherein the plastic material is prepared by obtaining at least one of virgin polymeric material from the group consisting of polyolefin, polyacetal, polyamide, polyester, cellulose ether and ester, polyalkylene sulfide, polyarylene oxide, polysulfone, modified polysulfone polymers and mixtures thereof.

15. The method of making odor controlled bio-composite of claim 12 wherein the biological material is processed by a hydrolysis method comprising steps of:
reducing a biological material to a particle size appropriate for a hydrolysis process;
drying the sized biological materials to a moisture content less than 25% by weight;
hydrolyzing the dried biological material in a pressurized hydrothermal vessel by subjecting the biological material with steam; and
drying the hydrolyzed biological material to a moisture content less than 15% by weight.

16. The method of making odor controlled bio-composite of claim 12 wherein the biological material comprises at least one byproduct of energy production generate from at least one of the group consisting of corn, soybean, flaxseed, switchgrass, rapeseed, miscanthus, hulls, stover, straw, bagasse from sugarcane and jatropha processed using a hydrolysis system.

17. The method of making odor controlled bio-composite of claim 12 wherein the biological material comprises at least one of the group consisting of corn, soybean, wheat, barley, oats, sorghum(milo), sunflower, safflower, buckwheat, flax, peanut, rice, cnola, rye, millet, triticale, chickpeas, lentils, and filed peas.

18. The method of making odor controlled bio-composite of claim 12 wherein the biological material is DDG and DDG is processed by a classification method comprising steps of:
classifying DDG to separate a fiber material from a non-fiber material; and
selecting the classified fiber material according to as desired composite property.

19. The method of making odor controlled bio-composite of claim 12 wherein the biological material is process by a classification method comprising steps of:
grinding the biological material;
classifying the biological material to separate a fiber material from a non-fiber material; and
selecting the classified fiber material according to as desired composite property.

20. The method of making odor controlled bio-composite of claim 12 wherein the odor controlling agent is a polymeric material carrying at least one of a fragrance and an odor neutralizer, the polymeric material different than said plastic material, the polymeric material integrated with said plastic material.

21. The method of making odor controlled bio-composite of claim 20 wherein said polymeric material is EVA.

22. The method of making odor controlled bio-composite of claim 12 wherein said odor controlling agent is an odor reducing agent comprising at least one of activated carbon, activated anthracite, ziolite, silica gel, and baking soda.

23. The method of making odor controlled bio-composite of claim 12 wherein the biological material is first integrated with the plastic material to form a bio-plastic composite, then integrating the odor controlling agent with the bio-plastic composite to form said odor controlled bio-plastic composite.

24. The method of making odor controlled bio-composite of claim 23 wherein the odor controlling agent is integrated with the bio-plastic composite by dipping the bio-plastic composite in a pan containing at least one of a fragrance and a odor neutralizer.

25. The method of making odor controlled bio-composite of claim 23 wherein the odor controlling agent is integrated with the bio-plastic composite by spraying the bio-plastic composite with at least one of a fragrance and a odor neutralizer.

26. A bio-plastic composite comprising:
a plastic material; and
at least one member of the group consisting of
(a) a hydrolyzed biological material, amount up to 99% by weight, integrated with the plastic material;
(b) a classified biological material, amount up to 50% by weight, integrated with the plastic material; and
(c) a cryogenically ground material including at least one biological material integrated with the plastic material.

27. The bio-plastic composite of claim 26 wherein said cryogenically ground material includes at least one of a recycled tire material and a recycled high temperature plastic material, and at least one biological material.

28. A method of making bio-plastic composite pellet comprising the steps of:
preparing a plastic material;
processing a biological material;
mixing the plastic material and the biological material;
extruding the mixture by feeding the mixture into an extruder with a barrel heated according to a melting temperature of the plastic material;
removing contaminants from the mixture by passing the molten mixture through a screen pack located in the front section of the barrel; and
forming bio-plastic composite pellets in a die.

29. The method of claim 28, further comprising the step of:
feeding the pellets into a injection molding machine wherein the pellets are melted and molded in a mold.

30. The method of claim 28, further comprising the step of:
feeding the pellets into a injection blow molding machine where the pellets are melted and injected through a nozzle into a preformed hollow mold which determines an outer shape of an end product.

31. The method of claim 28, further comprising the steps of:
making second bio-plastic composite pellets with a different combination of a plastic material and a biological material; and
feeding bio-plastic pellets into first extruder and second bio-plastic pellets into second extruder, and simultaneously running first and second extruders at a steady volumetric throughput to a single extrusion head which combines first layer of bio-plastic composite and second layer of bio-plastic composite in to a desired shape.

32. The method of making bio-plastic composite of claim 31 wherein the thickness of first layer of bio-plastic composite and second layer of bio-plastic composite are controlled by a relative speed of first extruder and second extruder.

33. The method of making bio-plastic composite of claim 31 wherein a third layer of bio-plastic composite of different formula is extruded to the extrusion head.

34. A method of making bio-composite comprising the steps of:
preparing a plastic material;
processing a biological material;
mixing the plastic material and the biological material;
extruding the mixture by feeding the mixture into an extruder with a barrel heated according to melting temperature of the plastic material;
removing contaminants from the mixture by passing the molten mixture through a screen pack located in the front section of the barrel;
forcing the molten mixture through a die adjusted to obtain a bio-composite sheet stock with a desired thickness;
reheating the bio-composite sheet stock until soft; and
forming the soft sheet stock in a mold to a desired shape.

35. A concrete mixture comprising:
a cement material;
a water; and
a bio-plastic composite material.

36. A method of making concrete comprising the steps of:
making a bio-plastic composite material;
mixing a cement material and a water;
integrating the bio-plastic composite material with the cement and water mixture;
applying the bio-plastic composite cement mixture; and
drying to cure the mixture into a concrete.
